(19) United States
(12) Patent Application Publication
(10) Pub. No.: US 2011/0087287 A1
(43) Pub. Date: Apr. 14, 2011

(54) Rod-to-Rod Connector

(75) Inventors: Ralph F. Reeder, JR., Dakota Dunes, SD (US); Quentin Durward, North Sioux City, SD (US); Ahmad Faizan, Randolph, NJ (US); Mahmoud F. Abdelgany, Rockaway, NJ (US)

(73) Assignee: CUSTOM SPINE, INC., Parsippany, NJ (US)

(21) Appl. No.: 12/576,929

(22) Filed: Oct. 9, 2009

(51) Int. Cl. A61B 17/70

(52) U.S. Cl. 606/250; 606/264

(57) ABSTRACT

A rod-to-rod connector assembly comprising a longitudinal member, which may be straight or S-shaped or have some other configuration; and a connecting member connected to a first end of the longitudinal member, wherein the connecting member comprises a pair of threaded opposed ends defining a channel therebetween. The assembly may further comprise a threaded blocking mechanism that engages the pair of threaded opposed ends of the connecting member. Preferably, the longitudinal member connects to only one of the opposed ends of the connecting member. The longitudinal member may comprise a first elongated portion comprising a first longitudinal axis; a second elongated portion comprising a second longitudinal axis; and a curved portion connecting the first elongated portion to the second elongated portion, wherein the first longitudinal axis and the second longitudinal axis are parallel to one another.

### BACKGROUND

1. Technical Field

The embodiments herein generally relate to medical devices, and, more particularly, to medical implant devices used in spinal surgeries.

2. Description of the Related Art

Lumbar fusion using pedicle screw rod systems is a state of the art surgical procedure used to treat various conditions in spine. An existing spinal construct is often extended upward to adjacent levels to treat the disease progressed to those levels. In order to create a continuous construct, the rods are connected. The general procedure includes removal of the blocker screw from the cranial screw. Subsequently, the screw is removed by bending the rod. Finally, the rod is bent back to normal position and a connector is used to bridge the previous rod and the rod from the new construct. The procedure is not only time consuming but is also prone to errors and could also potentially damage the rod during bending.

### SUMMARY

In view of the foregoing, an embodiment herein provides a rod-to-rod connector assembly comprising a longitudinal member, which may be straight or S-shaped or have some other configuration; and a connecting member connected to a first end of the longitudinal member, wherein the connecting member comprises a pair of threaded opposed ends defining a channel therebetween. The assembly may further comprise a threaded blocking mechanism that engages the pair of threaded opposed ends of the connecting member. Preferably, the longitudinal member connects to only one of the opposed ends of the connecting member. The longitudinal member may comprise a first elongated portion comprising a first longitudinal axis; a second elongated portion comprising a second longitudinal axis; and a curved portion connecting the first elongated portion to the second elongated portion, wherein the first longitudinal axis and the second longitudinal axis are parallel to one another.

The assembly may further comprise a first polyaxial pedicle screw assembly operatively connected to a second end of the longitudinal member, wherein the second end of the longitudinal member is positioned opposite to the first end of the longitudinal member; an elongated longitudinal member positioned through the channel of the connecting member; a second polyaxial pedicle screw assembly connected to a first end of the elongated longitudinal member; and a third polyaxial pedicle screw assembly connected to a second end of the elongated longitudinal member.

Each of the first polyaxial pedicle screw assembly, the second polyaxial screw assembly, and the third polyaxial screw assembly may comprise a bone fixator component comprising an open socket concave head portion; a screw head component comprising a expandable bulbous end engaging the head portion of the bone fixator component; and a pair of opposed ends defining a channel therebetween, wherein the elongated longitudinal member is positioned through the channel of each of the second polyaxial pedicle screw assembly and the third polyaxial pedicle screw assembly. The assemblies may further comprise a blocker mechanism engaging the screw head component.

One of the threaded opposed ends of the connecting member may comprise a block portion comprising a first bore intersecting a second bore, wherein the second bore is positioned parallel to the channel. The assembly may further comprise a threaded blocking mechanism that engages the first bore of the block portion of the connecting member. The first end of the longitudinal member preferably connects to the second bore of the block portion of the connecting member.

These and other aspects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

### BRIEF DESCRIPTION OF THE DRAWINGS

The embodiments herein will be better understood from the following detailed description with reference to the drawings, in which:

FIG. 1A is a perspective view of a left-side rod-to-rod connector implant assembly according to a first embodiment herein;

FIG. 1B is a perspective view of a right-side rod-to-rod connector implant assembly according to a first embodiment herein;

FIG. 2A is a top view of a left-side rod-to-rod connector implant according to a first embodiment herein;

FIG. 2B is a top view of a right-side rod-to-rod connector implant according to a first embodiment herein;

FIG. 3A is a perspective view of a left-side rod-to-rod connector implant apparatus according to a first embodiment herein;

FIG. 3B is a top view of a left-side rod-to-rod connector implant apparatus according to a first embodiment herein;

FIG. 4A is a perspective view of a connector component of a rod-to-rod connector implant according to a second embodiment herein;

FIG. 4B is a perspective view of a connector and blocker screw component of a rod-to-rod connector implant according to a second embodiment herein;

FIG. 5A is a perspective view of a left-side rod-to-rod connector implant apparatus according to a second embodiment herein;

FIG. 5B is a top view of a left-side rod-to-rod connector implant apparatus according to a second embodiment herein;

FIG. 6 is a top view of a right-side rod-to-rod connector implant apparatus according to a third embodiment herein;

FIG. 7 is a top view of a straight rod-to-rod connector implant apparatus according to a fourth embodiment herein;

FIG. 8A through 8G illustrate various views of a screw head component of a polyaxial pedicle screw assembly used in accordance with the embodiments herein;

FIGS. 9A and 9B illustrate a first configuration of a saddle pin used in accordance with the embodiments herein;

FIGS. 10A and 10B illustrate a second configuration of a saddle pin used in accordance with the embodiments herein;

FIG. 11 illustrates a third configuration of a saddle pin used in accordance with the embodiments herein; and

FIG. 12 illustrates an isolated view of the end of one configuration of a saddle pin used in accordance with the embodiments herein.

### DETAILED DESCRIPTION OF PREFERRED EMBODIMENTS

The embodiments herein and the various features and advantageous details thereof are explained more fully with reference to the non-limiting embodiments that are illustrated in the accompanying drawings and detailed in the following description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

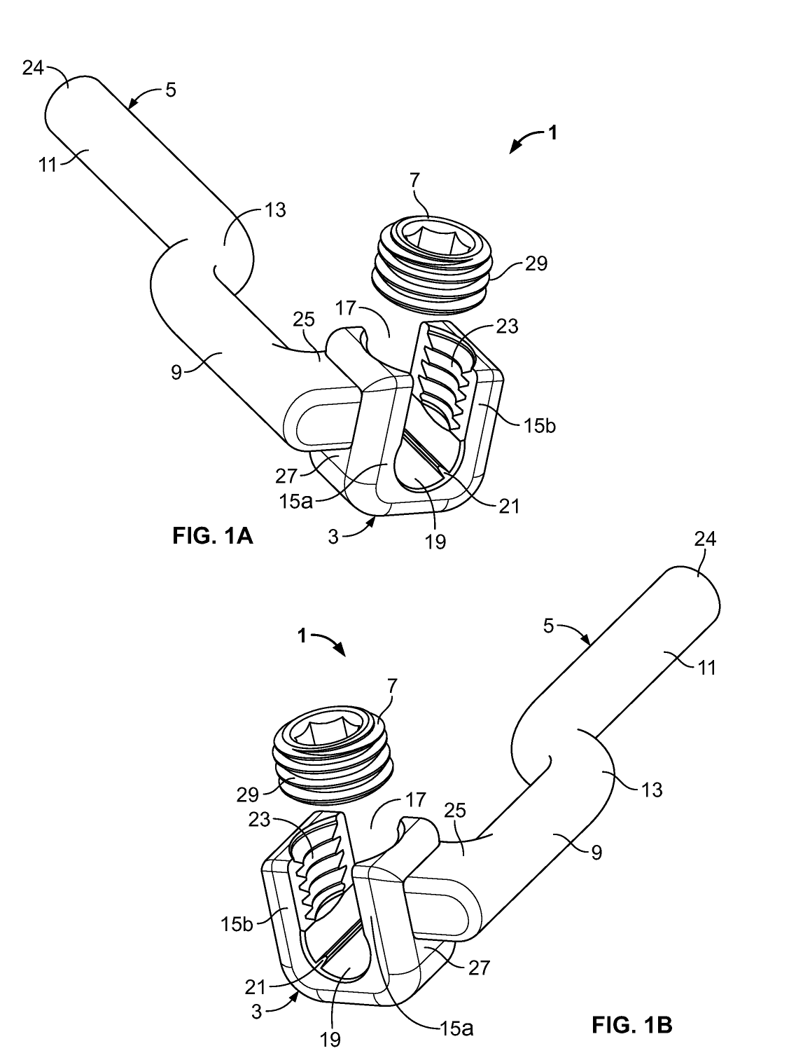

The embodiments herein provide a medical implant device to extend a pedicle screw rod system in the spine without having to remove the existing screws. The device may be used to connect two spinal rods longitudinally. Referring now to the drawings, and more particularly to FIGS. 1A through 12, where similar reference characters denote corresponding features consistently throughout the figures, there are shown preferred embodiments.

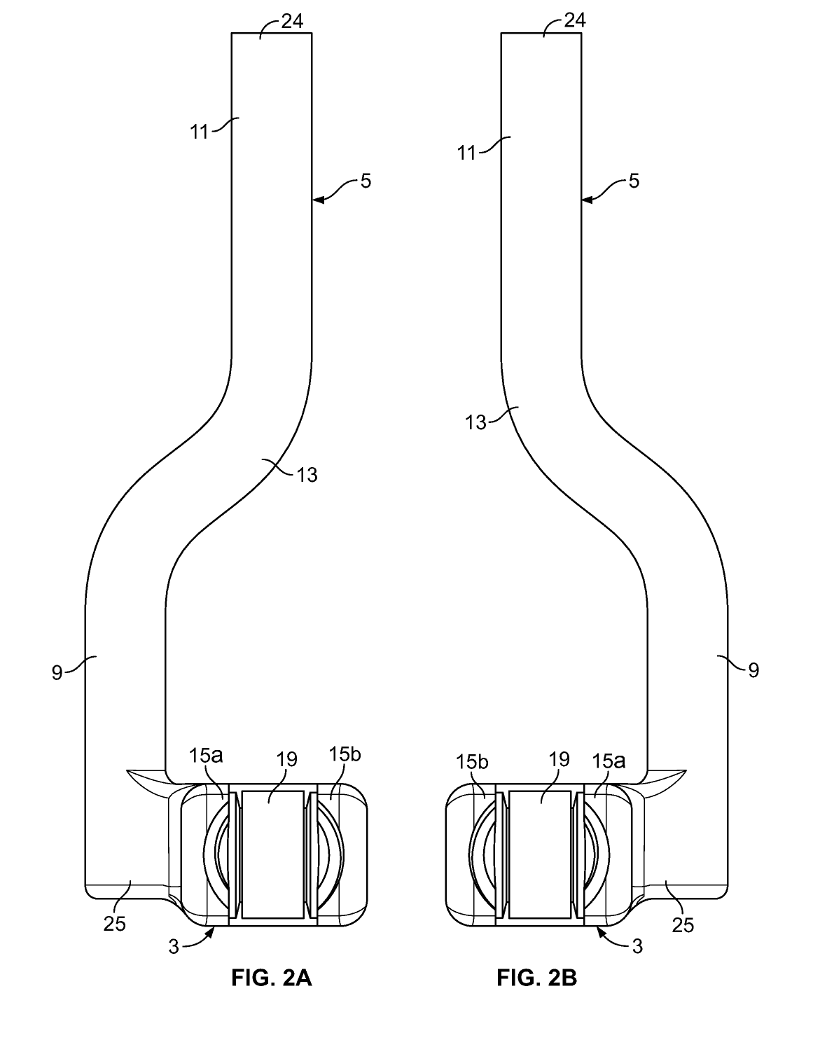

FIGS. 1A through 2B illustrate a rod-to-rod connector implant assembly 1 according to a first embodiment herein. FIGS. 1A and 2A illustrate a left-side configuration while FIGS. 1B and 2B illustrate a right-side configuration. The rod-to-rod connector assembly 1 comprises a longitudinal member 5 and a connecting member 3 connected to a first end 25 of the longitudinal member 5, wherein the connecting member 3 comprises a pair of threaded opposed ends 15a, 15b defining a channel 17 therebetween. While shown in an S-shaped configuration, the longitudinal member 5 may be configured in any dimension or configuration and the embodiments herein are not restricted to any particular geometry, configuration, shape, or design.

The first end 25 of the longitudinal member 5 connects to outer wall 27 of the opposed end 15a of the connecting member 3. Furthermore, the channel 17 is generally U-shaped comprising a bottom portion 19 with grooves 21. The connecting member comprises threads 23 configured along the inner walls of the opposed ends 15a, 15b. The assembly 1 further comprises a threaded blocking mechanism 7 that engages the pair of threaded opposed ends 15a, 15b of the connecting member 3. The longitudinal member 5 connects to only one of the opposed ends 15a of the connecting member 3.

The longitudinal member 5 comprises a first elongated portion 9 comprising a first longitudinal axis; a second elongated portion 11 comprising a second longitudinal axis; and a curved portion 13 connecting the first elongated portion 9 to the second elongated portion 11, wherein the first longitudinal axis and the second longitudinal axis are parallel to one another.

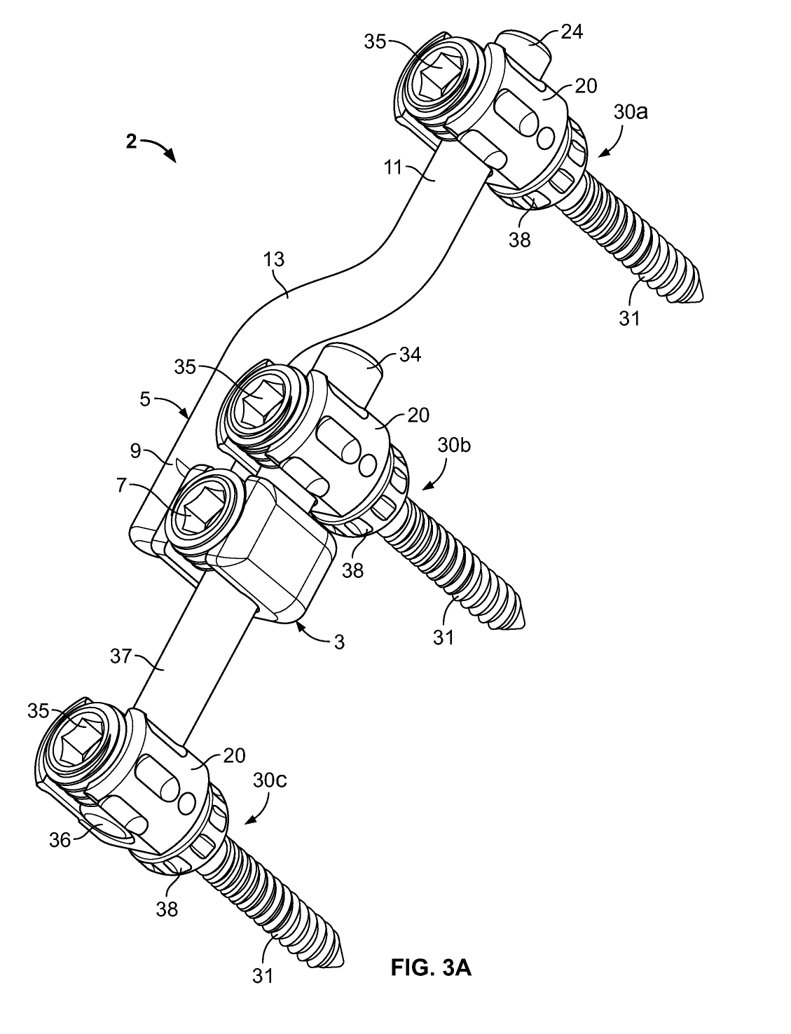

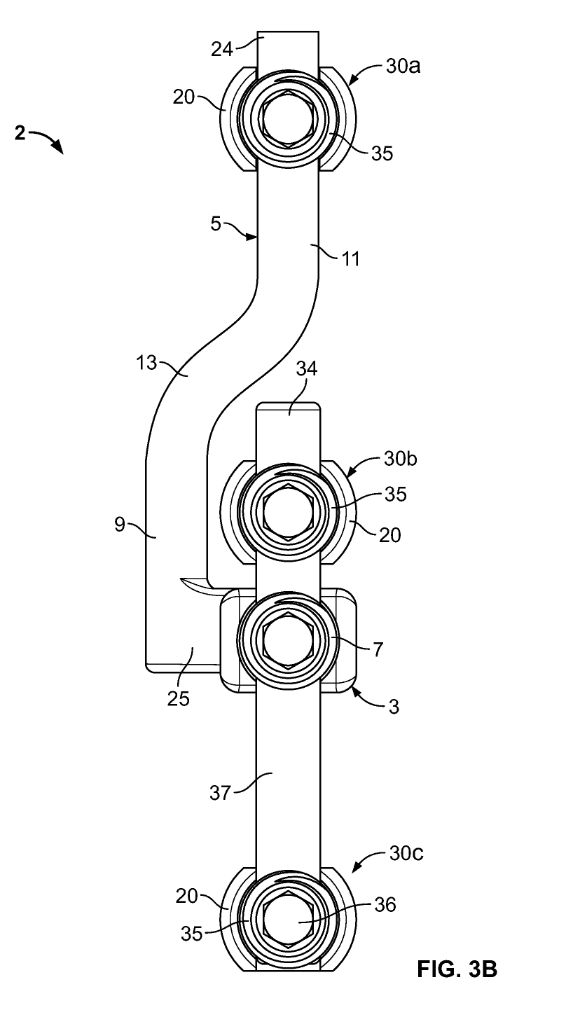

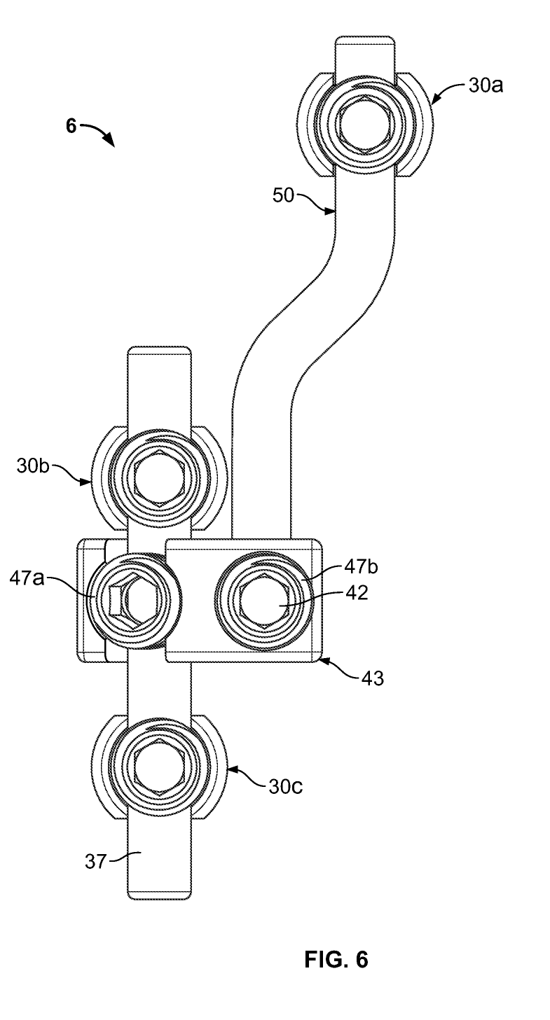

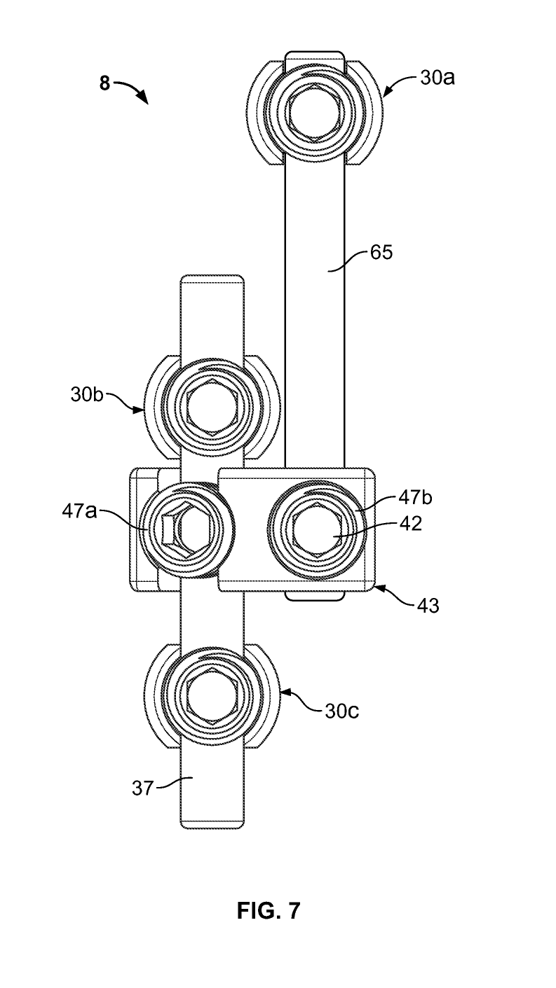

FIGS. 3A and 3B, with reference to FIGS. 1A through 2B, illustrate a left-side rod-to-rod connector implant apparatus 2 according to a first embodiment herein. The rod-to-rod connector apparatus 2 provided by the embodiments herein avoids both the removal of the existing screw constructs 30b, 30c and the rod 37 bending action. The channel 17 of the connecting member is attached to the existing rod 37 with the help of blocker 7. The blocker 7 comprises threads 29 configured around the outer perimeter of the blocker 7 (although other configurations are possible) to secure the rod 37 in place. The longitudinal member 5 protruding from the connecting member 3 is accepted by the screw head 20 of the new pedicle screw assembly construct 30a. The protruding longitudinal member 5 may either be S-shaped (as shown in FIGS. 1A through 3B and 5A through 6) or a straight rod (as shown in FIG. 7) depending on the lateral offset distance between the screw assemblies 30a, 30b, 30c being connected together.

The apparatus 2 further comprises a first polyaxial pedicle screw assembly 30a operatively connected to a second end 24 of the longitudinal member 5, wherein the second end 24 of the longitudinal member is positioned opposite to the first end 25 of the longitudinal member 5; an elongated longitudinal member 37 positioned through the channel 17 of the connecting member 3; a second polyaxial pedicle screw assembly 30b connected to a first end 34 of the elongated longitudinal member 37; and a third polyaxial pedicle screw assembly 30c connected to a second end 36 of the elongated longitudinal member 37.

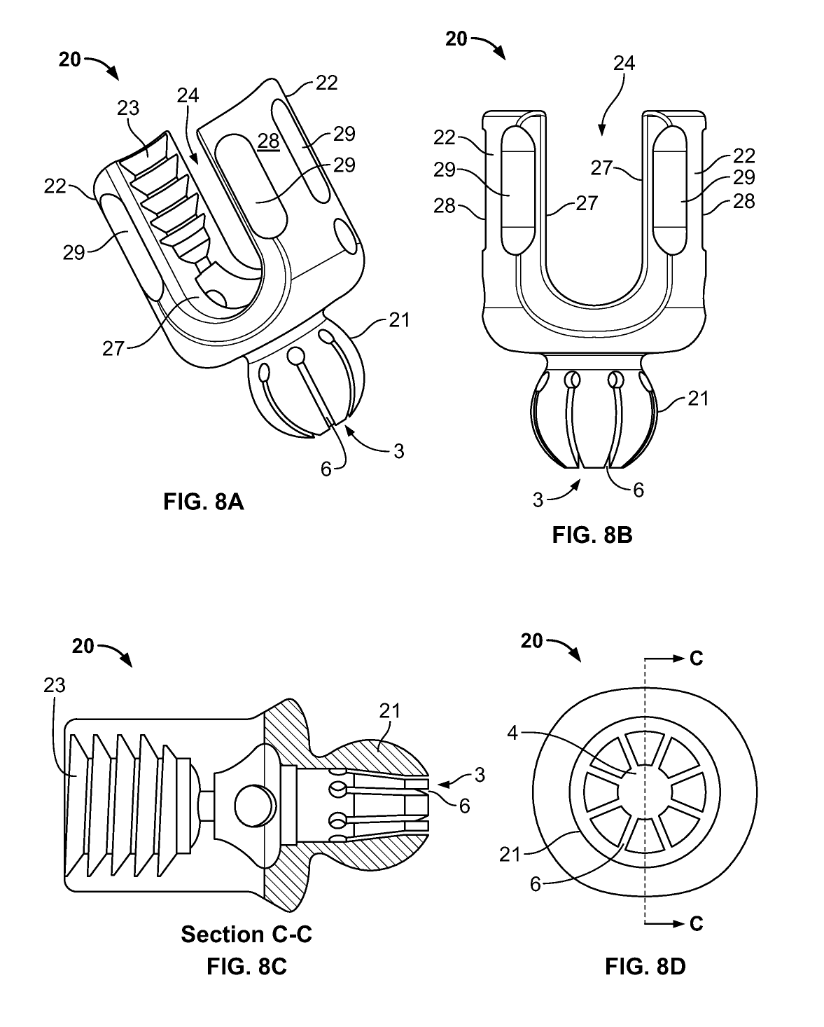

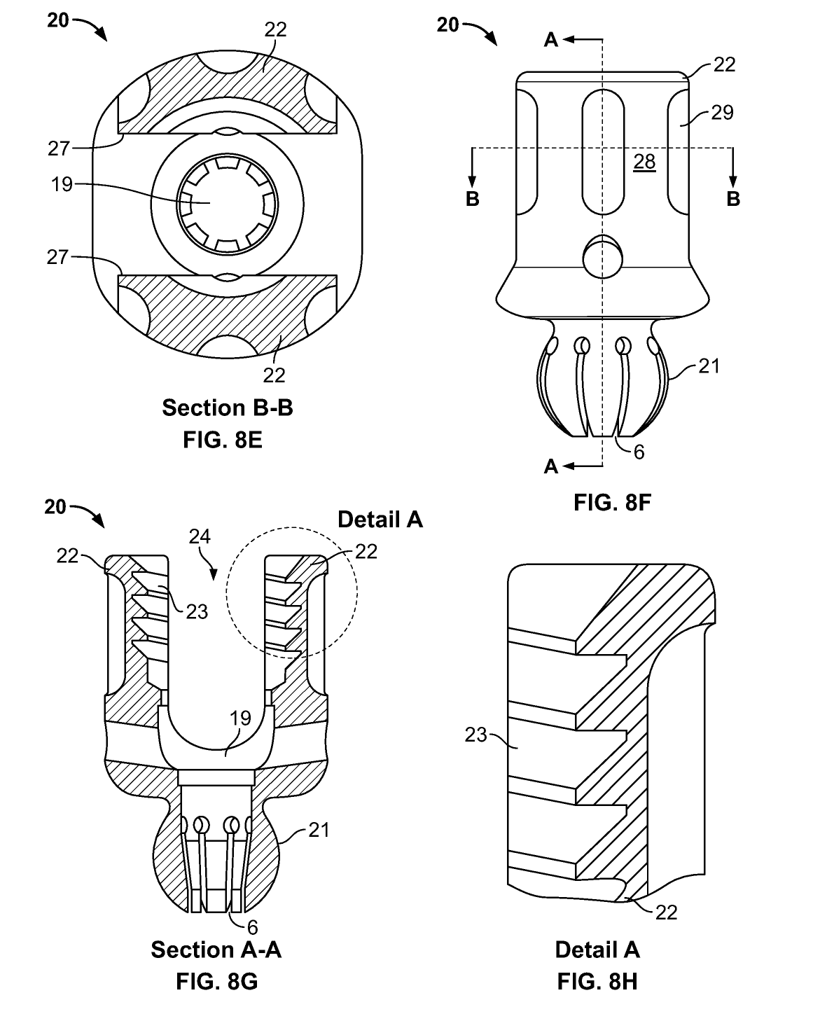

As further shown in FIG. 3A, each of the first polyaxial pedicle screw assembly 30a, the second polyaxial screw assembly 30b, and the third polyaxial screw assembly 30c comprises a bone fixator component 31 comprising an open socket concave head portion 38; a screw head component 20 comprising a expandable bulbous end 121 (shown in FIGS. 8A through 8G) engaging the head portion 38 of the bone fixator component 31; and a pair of opposed ends 122 (shown in FIGS. 8A through 8G) defining a channel 124 (shown in FIGS. 8A through 8G) therebetween, wherein the elongated longitudinal member 37 is positioned through the channel 124 of each of the second polyaxial pedicle screw assembly 30b and the third polyaxial pedicle screw assembly 30c. A threaded blocker mechanism 35 engages the screw head component 20. The blocker 35 is dimensioned and configured to mate with the wall threads 123 of the screw head 20.

In operation, the existing construct comprising assemblies 30b, 30c and rod 37 are already set in place in bone (i.e., in the spine (not shown)). Then, assembly 1 is connected to the rod 37 by seating the rod 37 in the channel 17 of the connecting member 3. Then, a new screw assembly 30a is set in place in an adjacent bone (not shown) and after the longitudinal member 5 is seated in the screw head 20 of the assembly 30a, the blocker mechanism 35 is applied to the assembly 30a along with the blocker mechanism 7 to the connecting member 3. This allows the assemblies 30a, 30b, 30c to be operatively connected together using connecting member 3 and longitudinal member 5.

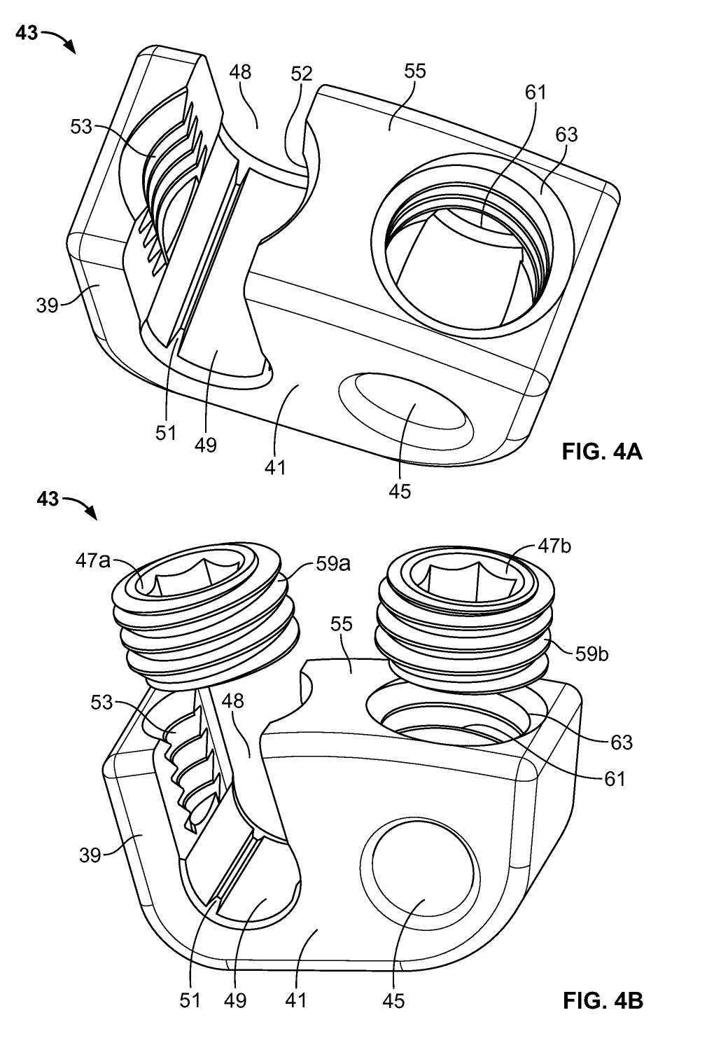

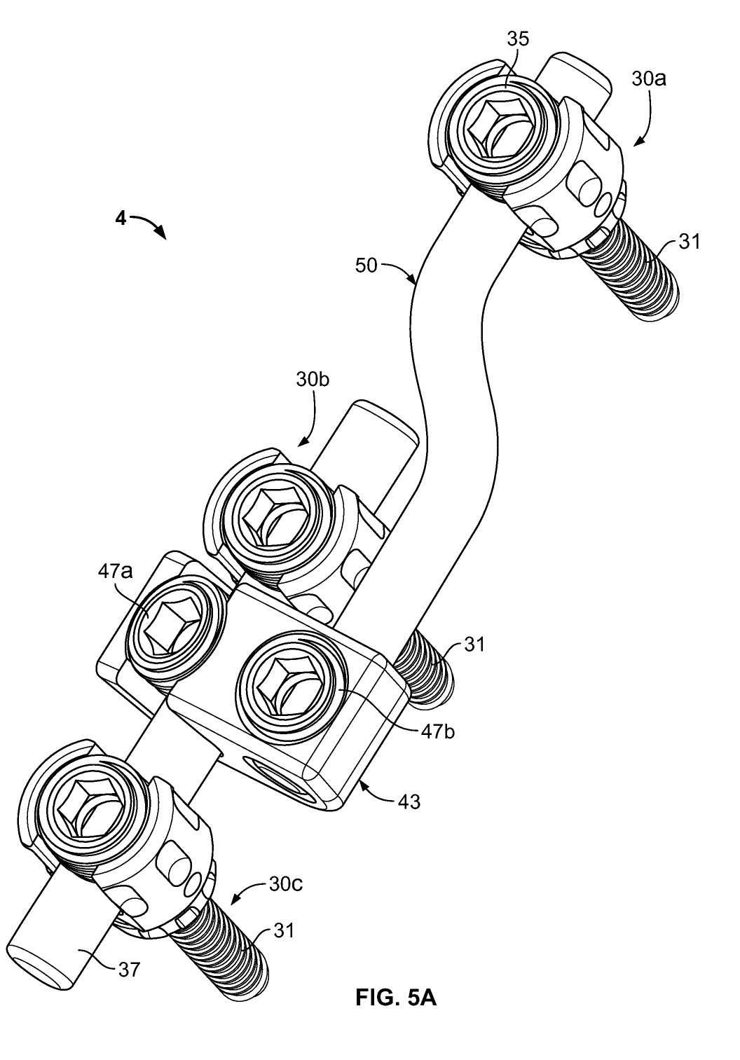

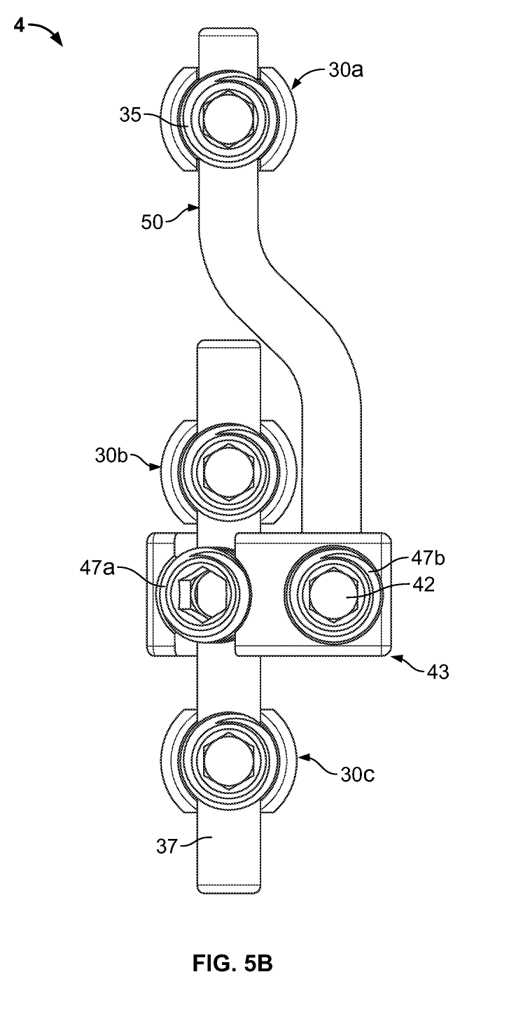

FIGS. 4A and 4B illustrate a connector component 43 according to a second embodiment herein. FIGS. 5A and 5B illustrate a left-side rod-to-rod connector implant apparatus 4 according to a second embodiment herein. In this embodiment, the connector component 43 comprises a block portion 55 comprising a first bore 61 intersecting a second bore 45, wherein the second bore 45 is positioned parallel to the channel 48. A threaded end 39 is spaced apart from the block portion 55, which defines a channel 48 therebetween. The channel comprises a generally U-shaped wall that has a bottom portion 49 comprising grooves 51 and accommodates a longitudinal member 37 to rest therein. The threaded end 39 comprises threads 53 that match threads on an opposite wall 52 adjacent to the block portion 55. The threads 53 and threaded wall 52 mate with the threads 59a of a corresponding threaded blocker mechanism 47a. A blocking mechanism 47b comprising threads 59b engages the threads 63 of the first bore 61 of the block portion 55 of the connecting member 43. The first end 42 of the longitudinal member 50 connects to the second bore 45 of the block portion 55 of the connecting member 43 with the blocking mechanism 47b locking the longitudinal member 50 in place.

In operation, the existing construct comprising assemblies 30b, 30c and rod 37 are already set in place in bone (i.e., in the spine (not shown)). Then, connecting member 43 is connected to the rod 37 by seating the rod 37 in the channel 48 of the connecting member 43. After this, the first end 42 of the longitudinal member 50 is inserted in the second bore 45 of the connecting member 43. Then, a new screw assembly 30a is set in place in an adjacent bone and after the longitudinal member 50 is seated in the screw head 20 of the assembly 30a, the blocker mechanism 35 is applied to the assembly 30a along with the blocker mechanism 47b to the connecting member 3. This allows the assemblies 30a, 30b, 30c to be operatively connected together using connecting member 43 and longitudinal member 50.

FIG. 6 is a top view of a right-side rod-to-rod connector implant apparatus 6 according to a third embodiment herein. The configuration is similar to the apparatus 4 of FIGS. 5A and 5B except that the longitudinal member 50 is turned to the right. FIG. 7 is a top view of a straight rod-to-rod connector implant apparatus 8 according to a fourth embodiment herein. The configuration is similar to the apparatus 4 of FIGS. 5A and 5B and the apparatus 6 of FIG. 6 except that the longitudinal member 65 is straight (i.e., has one longitudinal axis without any curves) and similarly configured to the longitudinal member 37.

FIGS. 8A through 12 illustrate an example of a pedicle screw system comprising a screw head and saddle pin, which could be used in accordance with the embodiments herein. Those skilled in the art would readily understand that the examples shown in FIGS. 8A through 12 as well as the configuration shown for the bone screw 31 shown in FIGS. 3A and 5A are merely for illustrative purposes and that the embodiments herein are not limited to any particular type of pedicle screw assembly which may constitute the assemblies 30a, 30b, 30c. Indeed any type/configuration of pedicle screw assembly may be used in accordance with the embodiments herein.

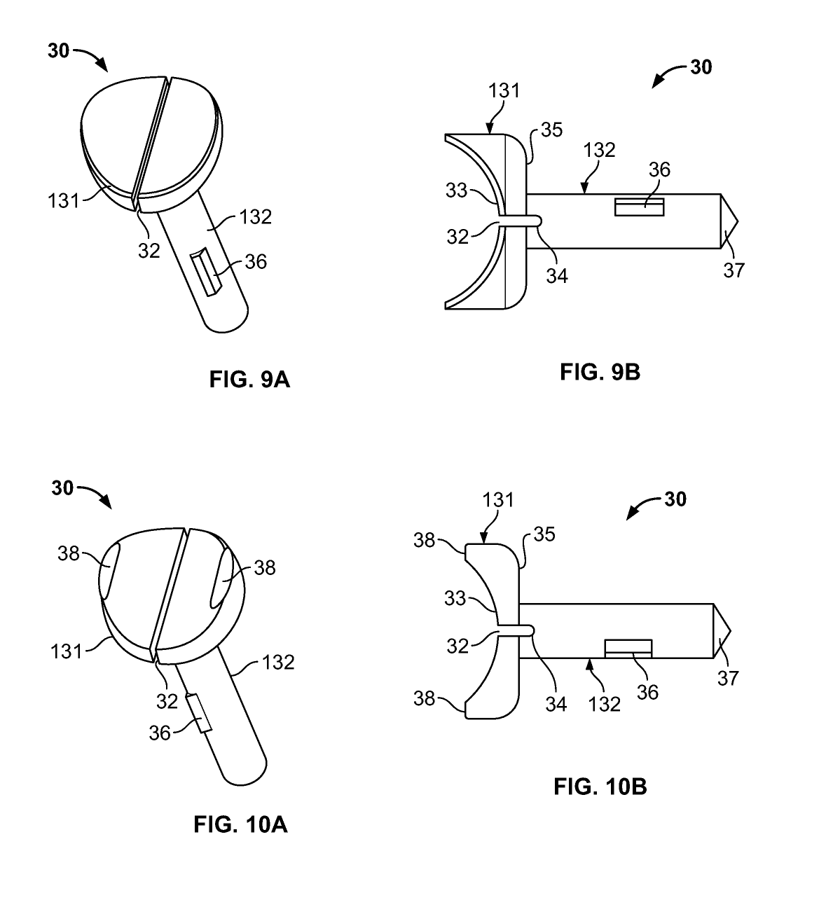

FIG. 8A illustrates the overall configuration of the screw head 20. FIG. 8B illustrates a front view of the screw head 20. FIG. 8C is a cross-sectional view from cut-line “CC” of FIG. 8D. FIG. 8E is a cross-sectional view from cut-line “BB” of FIG. 8F and FIG. 8G is a cross-sectional view from cut-line “AA” of FIG. 8F. Additionally, FIG. 8H is an enlarged detailed view of the encircled area “A” of FIG. 8G illustrating the threaded inner portion 123 in more detail. As shown in FIGS. 9A through 9H, the screw head 20 includes a tip portion 103 comprising a bulbous (spherical) male end 121 for engaging the concave female socket 112 of the bone screw. The screw head 20 also includes a pair of upright ends 122 opposite the bulbous male end 121, wherein the upright ends 122 comprise a threaded inner portion 123 for engaging the blocker 35. Furthermore, the screw head 20 includes a generally open U-shaped inner portion 124 for receiving the saddle pin 130 and the longitudinal member 5, 37, 50, 65. The male end 121 of the screw head 20 includes a plurality (for example, four or more) slots 116 forming a central hole 104 that allow the male end 121 to expand into the female spherical pocket 38 of the bone screw 31 at any allowable angle once the saddle pin 130 is forced through.

The screw head 20 further comprises two opposed upright ends 122 separated by the slot 124, wherein each of the opposed upright ends 122 comprise an inner wall 127 and an outer wall 128, wherein the inner wall 127 comprises wall threads 123, and wherein the outer wall 128 comprises grooves (cuts) 129. The upper saddle portion 131 of the pin 130 comprises a slot 132. The bulbous end 121 of the screw head 20 comprises a plurality of slots 116 terminating at hole 104 at a tip 103 of the bulbous end 121. Moreover, the hole 104 is configured to receive the pin 130.

Since the screw head 20 is pivoting inside the female socket end 112 of the bone screw 31, the assembly 30a, 30b, 30c is allowed to be inserted deeper into the bone without having the bone or anatomy prematurely limit the range of angulations of the screw head 20. The screw head 20 further includes external features or cuts 129 that assist in accommodating surgical instrumentation during manipulation and assembly during the surgical procedure. These cuts 129 allow various instruments (not shown) to firmly and positively hold and manipulate the screw head 20 on one side or both sides of screw head 20.

The several embodiments of the saddle pin 130 are shown in FIG. 9A through FIG. 12. The saddle pin 130 provides a proper seat for the longitudinal member 5, 37, 50, 65 and avoids notching a typical titanium longitudinal member 5, 37, 50, 65 (titanium is very notch sensitive). Furthermore, the saddle pin 130 allows one to accommodate multiple sizes of longitudinal members 5, 37, 50, 65 in the same screw assembly system 30a, 30b, 30c. The saddle pin 130 is configured with a slot 132 through the center to allow expansion of the upper portion (head) 1131 of the saddle pin 130. The bottom 135 of the saddle pin head 1131 is angled to accommodate the saddle pin 130 when spreading to accept a larger-sized longitudinal member 5, 37, 50, 65. The saddle pin 130 initially expands the male sphere 121 of the screw head 20 into the female spherical socket 38 in the bone screw 31 causing the screw assembly system 30a, 30b, 30c to lock or start locking (i.e., causing the male sphere 121 of the screw head 20 to lock in the female spherical socket 38 of the bone screw 31). The saddle pin 130 then “digs” into the female spherical socket 38 of the bone screw 31 to provide a secondary locking force to avoid bending failure of the assembly 30a, 30b, 30c.

FIGS. 9A through 9B illustrate a first embodiment of the saddle pin 130. The saddle pin 130 generally includes an upper portion 1131 and a lower portion 1132. The upper portion includes as slot 132, which is configured from the lowest area 133 of the upper portion 1131 into the upper area 134 of the lower portion 1132 of the saddle pin 130. A secondary locking mechanism 136 may be configured on the lower portion 1132 of the saddle pin to further achieve locking of the saddle pin 130 once it is inserted in the screw head 20. The lower portion 1132 of the saddle pin 130 terminates with a pointed end 137 to allow for digging into the female socket 38 of the bone screw 31. FIGS. 10A through 10B illustrate a second embodiment of the saddle pin 130. The difference between the first and second embodiments of the saddle pin 130 is that the upper portion of the saddle pin 1131 in the second embodiment includes two generally flat upper opposed ends 138 to more matingly configure with the geometry of the screw head 20 and the longitudinal member 5, 37, 50, 65.

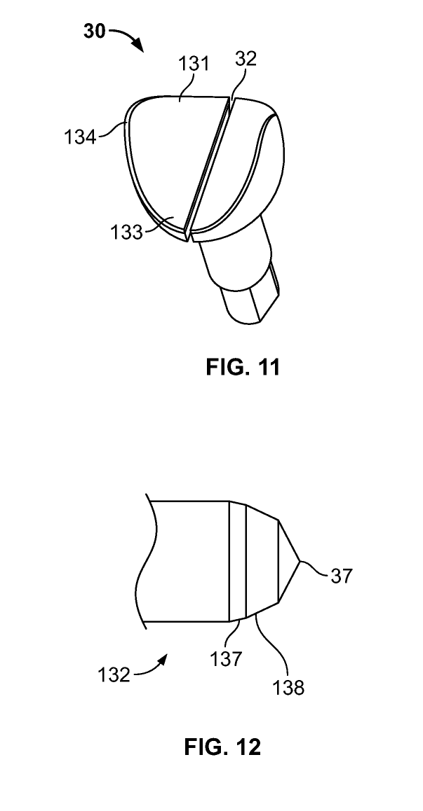

FIGS. 11 through 12 illustrate a third embodiment of the saddle pin 130. In particular, in the third embodiment, the saddle pin 130 comprises two parts: an upper portion 1131 preferably comprising titanium and a lower portion 1132 which is preferably ceramic. According to the third embodiment, the material of the lower portion 1132 of the saddle pin 130 is preferably ceramic and has a higher hardness and compressive yield strength than the comparative hardness and compressive yield strength of Ti₆Al₄V, which is the material which may be used in constructing the screw head 20 and bone screw 31.

As shown in FIG. 11, the upper portion 1131 of the saddle pin 130 includes a slot 132 in the seat portion 1133 and tapered angled ends 1134. Preferably, the saddle pin 130; i.e., the upper portion 1131 and the ceramic tip 1132 are assembled last in the overall process. Specifically, the screw head 20 snaps into the bone screw 31. Then, the ceramic tip 1132 slides into the screw head 20, and finally the titanium saddle (upper portion) 1131 is press fitted into the screw head 20 keeping everything in place and oriented in a relaxed state.

As best seen in FIG. 12, the lower portion 1132 of the saddle pin terminates with a series of cascading walls 1137, 1138 having sloped angles, terminating with the pointed end 137 for attachment into the screw head 20/bone screw 31 assembly. The material properties of the saddle pin tip 1134 are such that it prevents the deformation on the saddle pin 130 before the saddle pin 130 gives the proper bending and penetrating effects onto the screw head 20/bone screw 31 assembly. Examples of the types of materials used for the saddle pin pointed end 137 include Zyranox™ and HIP Vitox™, both of which are available from Morgan Advanced Ceramics, United Kingdom.

Accordingly, the pin 130 may comprise a multi-part assembly. The upper saddle portion 1131 of the pin 130 comprises titanium and the lower tip portion 1132 of the pin 130 comprises a ceramic material. Moreover, the lower tip portion 1132 comprises a mechanically harder material than the upper saddle portion 1131. The screw head 20 and the bone fixator component 31 comprise a first material, and the lower tip portion 1132 of the pin 130 comprises a material having a higher material hardness and compressive yield strength than the first material. Additionally, a wear resistant ceramic coating (not shown) may be configured over the screw head 20 and the bone fixator component 31.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the appended claims.

What is claimed is:

1. A rod-to-rod connector assembly comprising:
an S-shaped longitudinal member; and
a connecting member connected to a first end of said longitudinal member, wherein said connecting member comprises a pair of threaded opposed ends defining a channel therebetween.

2. The assembly of claim 1, further comprising a threaded blocking mechanism that engages said pair of threaded opposed ends of said connecting member.

3. The assembly of claim 1, wherein said longitudinal member connects to only one of the opposed ends of said connecting member.

4. The assembly of claim 1, wherein said longitudinal member comprises:
a first elongated portion comprising a first longitudinal axis;
a second elongated portion comprising a second longitudinal axis; and
a curved portion connecting said first elongated portion to said second elongated portion,
wherein said first longitudinal axis and said second longitudinal axis are parallel to one another.

5. The assembly of claim 1, further comprising:
a first polyaxial pedicle screw assembly operatively connected to a second end of said longitudinal member, wherein said second end of said longitudinal member is positioned opposite to said first end of said longitudinal member;
an elongated longitudinal member positioned through said channel of said connecting member;
a second polyaxial pedicle screw assembly connected to a first end of said elongated longitudinal member; and
a third polyaxial pedicle screw assembly connected to a second end of said elongated longitudinal member.

6. The assembly of claim 5, wherein each of said first polyaxial pedicle screw assembly, said second polyaxial screw assembly, and said third polyaxial screw assembly comprises:
a bone fixator component comprising an open socket concave head portion;
a screw head component comprising:
a expandable bulbous end engaging said head portion of said bone fixator component; and
a pair of opposed ends defining a channel therebetween, wherein said elongated longitudinal member is positioned through said channel of each of said second polyaxial pedicle screw assembly and said third polyaxial pedicle screw assembly; and
a blocker mechanism engaging said screw head component.

7. The assembly of claim 1, wherein one of said threaded opposed ends of said connecting member comprises a block portion comprising a first bore intersecting a second bore, wherein said second bore is positioned parallel to said channel.

8. The assembly of claim 7, further comprising a threaded blocking mechanism that engages said first bore of said block portion of said connecting member.

9. The assembly of claim 7, wherein said first end of said longitudinal member connects to said second bore of said block portion of said connecting member.

10. A medical implant device comprising:
a longitudinal member; and
a connecting member connected to a first end of said longitudinal member, wherein said connecting member comprises a pair of threaded opposed ends defining a channel therebetween.

11. The device of claim 10, further comprising a threaded blocking mechanism that engages said pair of threaded opposed ends of said connecting member.

12. The device of claim 10, wherein said longitudinal member connects to only one of the opposed ends of said connecting member.

13. The device of claim 10, wherein said longitudinal member comprises:
a first elongated portion comprising a first longitudinal axis;
a second elongated portion comprising a second longitudinal axis; and
a curved portion connecting said first elongated portion to said second elongated portion,
wherein said first longitudinal axis and said second longitudinal axis are parallel to one another.

14. The device of claim 10, further comprising:
a first polyaxial pedicle screw assembly operatively connected to a second end of said longitudinal member, wherein said second end of said longitudinal member is positioned opposite to said first end of said longitudinal member;
an elongated longitudinal member positioned through said channel of said connecting member;
a second polyaxial pedicle screw assembly connected to a first end of said elongated longitudinal member; and
a third polyaxial pedicle screw assembly connected to a second end of said elongated longitudinal member.

15. The device of claim 14, wherein each of said first polyaxial pedicle screw assembly, said second polyaxial screw assembly, and said third polyaxial screw assembly comprises:
a bone fixator component comprising an open socket concave head portion;
a screw head component comprising:
a expandable bulbous end engaging said head portion of said bone fixator component; and
a pair of opposed ends defining a channel therebetween, wherein said elongated longitudinal member is positioned through said channel of each of said second polyaxial pedicle screw assembly and said third polyaxial pedicle screw assembly; and
a blocker mechanism engaging said screw head component.

16. The device of claim 10, wherein one of said threaded opposed ends of said connecting member comprises a block portion comprising a first bore intersecting a second bore, wherein said second bore is positioned parallel to said channel, and wherein said first end of said longitudinal member connects to said second bore of said block portion of said connecting member.

17. The device of claim 16, further comprising a threaded blocking mechanism that engages said first bore of said block portion of said connecting member.

18. An apparatus comprising:
a longitudinal member comprising:
a first elongated portion comprising a first longitudinal axis;
a second elongated portion comprising a second longitudinal axis; and
a curved portion connecting said first elongated portion to said second elongated portion,
wherein said first longitudinal axis and said second longitudinal axis are parallel to one another;
a connecting member connected to a first end of said longitudinal member, wherein said connecting member comprises a pair of threaded opposed ends defining a channel therebetween, and wherein said longitudinal member connects to only one of the opposed ends of said connecting member;
a threaded blocking mechanism that engages said pair of threaded opposed ends of said connecting member;
a first polyaxial pedicle screw assembly operatively connected to a second end of said longitudinal member, wherein said second end of said longitudinal member is positioned opposite to said first end of said longitudinal member;
an elongated longitudinal member positioned through said channel of said connecting member;
a second polyaxial pedicle screw assembly connected to a first end of said elongated longitudinal member; and
a third polyaxial pedicle screw assembly connected to a second end of said elongated longitudinal member.

19. The apparatus of claim 18, wherein each of said first polyaxial pedicle screw assembly, said second polyaxial screw assembly, and said third polyaxial screw assembly comprises:
a bone fixator component comprising an open socket concave head portion;
a screw head component comprising:
a expandable bulbous end engaging said head portion of said bone fixator component; and
a pair of opposed ends defining a channel therebetween, wherein said elongated longitudinal member is positioned through said channel of each of said second polyaxial pedicle screw assembly and said third polyaxial pedicle screw assembly; and
a blocker mechanism engaging said screw head component.

20. The assembly of claim 19, wherein one of said threaded opposed ends of said connecting member comprises a block portion comprising a first bore intersecting a second bore, wherein said second bore is positioned parallel to said channel, wherein said first end of said longitudinal member connects to said second bore of said block portion of said connecting member.
